(19) United States
(12) Patent Application Publication
(10) Pub. No.: US 2018/0055682 A1
(43) Pub. Date: Mar. 1, 2018

(54) FACIAL INTEGRITY ORAL APPLIANCE

(71) Applicants: Christopher Jay Gillette, Battle Creek, MI (US); Timothy Charles Bandeen, Battle Creek, MI (US); Emily Bandeen, Battle Creek, MI (US)

(72) Inventors: Christopher Jay Gillette, Battle Creek, MI (US); Timothy Charles Bandeen, Battle Creek, MI (US); Emily Bandeen, Battle Creek, MI (US)

(21) Appl. No.: 15/245,471

(22) Filed: Aug. 24, 2016

(51) Int. Cl. A61F 5/56; A61C 7/36; A61M 16/00; A61M 16/04; A61M 16/06

(52) U.S. Cl. CPC A61F 5/566; A61C 7/36; A61M 16/06; A61M 16/0493; A61M 16/0066

(57) ABSTRACT

A facial integrity oral appliance is provided. The appliance includes a lower member formed to fit over lower teeth and an upper member formed to fit over upper teeth. The lower member and the upper member each include a plurality of tooth impressions for receiving respective teeth of a dentition. The members may further include a facial surface on one side thereof and a lingual surface on the opposite side thereof. A connector connects the upper and lower members together. The connector has a rubber elasticity.

### BACKGROUND OF THE INVENTION

The present invention relates to preventing leakage of a CPAP mask and, more particularly, to a device and method that maintains facial integrity, which prevents leakage of CPAP masks while sleeping.

CPAP, or continuous positive airway pressure, is a treatment that uses mild air pressure to keep the airways open. CPAP typically is used by people who have breathing problems, such as sleep apnea. CPAP Masks leak when a person looses muscle activity during sleep. Users become non compliant using their CPAP due to the leakage of the mask. Therefore, users are not getting treatment for their serious sleep disorder.

As can be seen, there is a need for a device and method of preventing leakage of a CPAP mask while sleeping.

### SUMMARY OF THE INVENTION

In one aspect of the present invention, a facial integrity oral appliance comprises: an upper member and a lower member, each of the upper member and lower member comprising: a plurality of tooth impressions for receiving respective teeth of a dentition and having a facial surface on one side thereof and a lingual surface on the opposite side thereof, and a connector connecting the upper member and the lower member together, wherein the connector comprises a rubber elasticity.

In another aspect of the present invention, a method of maintaining facial integrity while sleeping comprising the steps of: placing an upper member on upper teeth, wherein the upper member comprises a plurality of tooth impressions having a facial surface on one side thereof and a lingual surface on the opposite side thereof; placing a lower member on lower teeth, wherein the lower member comprises a plurality of tooth impressions having a facial surface on one side thereof and a lingual surface on the opposite side thereof; connecting the upper member and the lower member together by a connector comprising rubber elasticity.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention in storage;

FIG. 2 is an exploded view of an embodiment of the present invention;

FIG. 3 is a detail perspective view of taken from line 3-3 in FIG. 1;

FIG. 4 is a perspective view of an embodiment of the present invention in use; and

FIG. 5 is a flowchart illustrating a method of making the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a facial integrity oral appliance that prevents a CPAP from leaking. The appliance is custom fit to the patients bite for facial structure and may be held together by bands. The appliance keeps facial integrity to CPAP users over night that struggle with mask leaks and opening their mouth during sleep. The appliance further maintains the position of the teeth and face as it was when a patient puts the CPAP mask on at the beginning of the night, maintaining the muscle structure of the face to eliminate leaks. The present invention can be used as a standalone treatment for sleep apnea as well as in conjunction with the CPAP mask

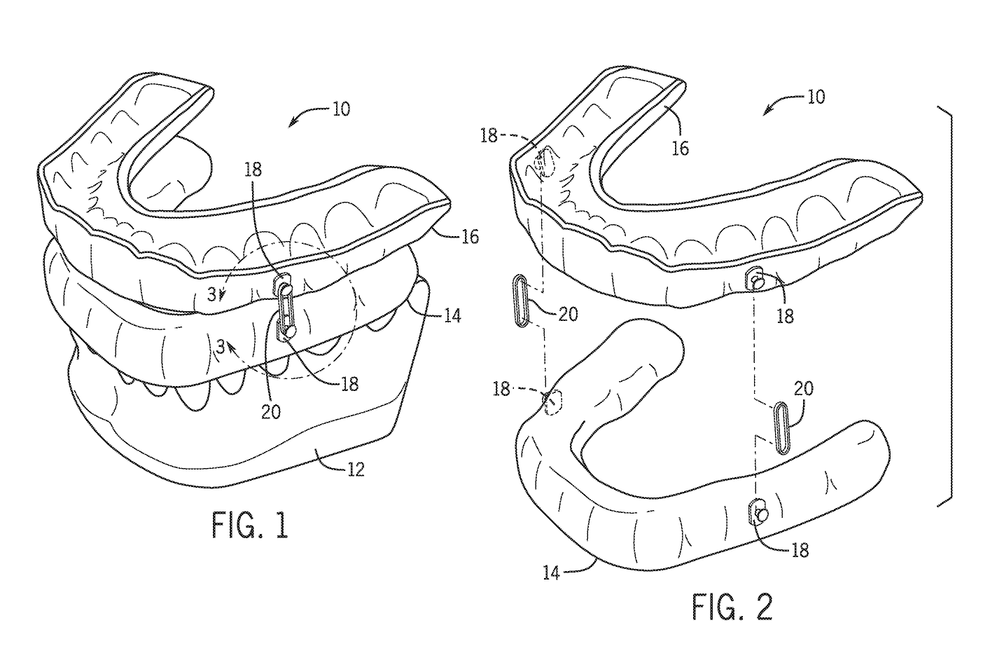

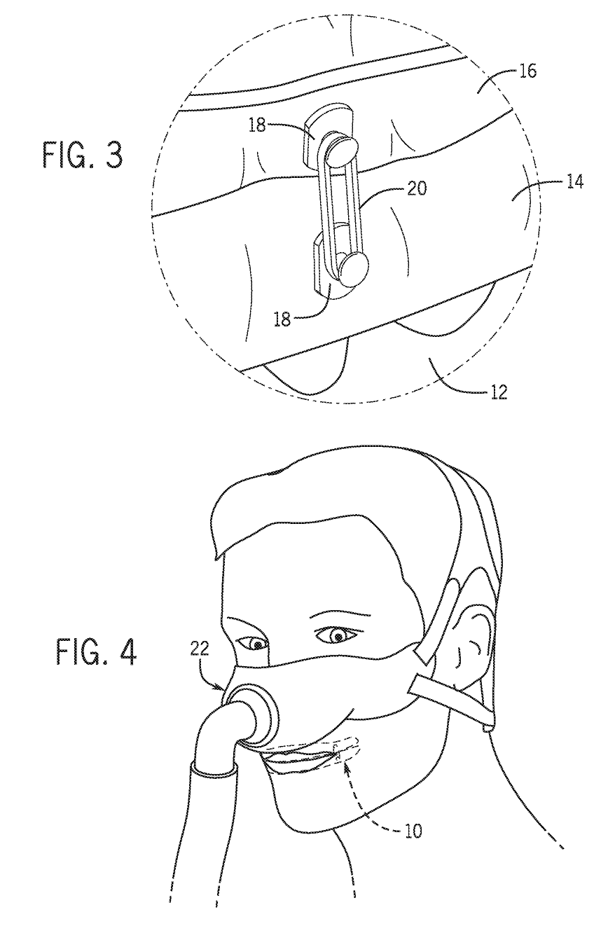

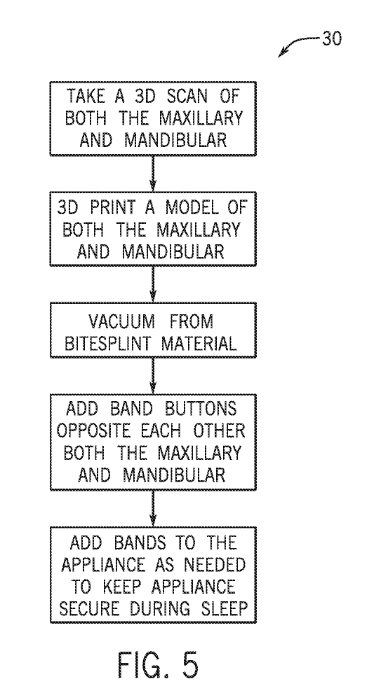

Referring to FIGS. 1 through 5, the present invention includes a facial integrity oral appliance 10. The appliance 10 includes a lower member 14 formed to fit over lower teeth and an upper member 16 formed to fit over upper teeth. The lower member 14 and the upper member 16 each include a plurality of tooth impressions for receiving respective teeth of a dentition. The members 14, 16 may further include a facial surface on one side thereof and a lingual surface on the opposite side thereof. A connector 20 connects the upper and lower members 14, 16 together. The connector 20 has a rubber elasticity.

The upper and lower members 14, 16 may be made of a clear plastic, such as a thermoplastic. The upper and lower members 14, 16 may be form fitted for a patient's teeth. The upper and lower members 14, 16 may further include a front portion for receiving incisal teeth and side portions for receiving occlusal teeth. Therefore, each of the upper and lower members 14, 16 may be U-shaped.

In certain embodiments, the lower member 14 and the upper member 16 may each include a first peg 18 and a second peg 18. The pegs 18 may include a base attached to the upper and lower members 16, a shaft protruding from the base, and a rounded flange formed at the end. The first peg 18 and the second peg 18 may protrude from an outer surface of the members 14, 16 facing away from the teeth. The first peg 18 may protrude from the facial surface of one of the side portions and the second peg 18 may protrude from the facial surface of the other of the side portions.

The connectors 20 of the present invention may include rubber bands. The rubber bands of the present invention may wrap around the pegs 18. For example, a first rubber band connects to the first peg 18 of the upper member 16 and the first peg 18 of the lower member 14 and the second rubber band connects to the second peg 18 of the upper member 16 and the second peg 18 of the lower member 14. The looped or endless rubber bands may wrap around the peg so that the rubber band is applying force against the shaft in between the base and the rounded flange. The rubber bands bias the upper member 16 and the lower member 14 together when worn, and thereby maintain facial integrity of the user.

A method of maintaining facial integrity while sleeping may include the steps of: placing the upper member 16 on upper teeth, placing the lower member 14 on lower teeth; and connecting the upper member 16 and the lower member 14 together by the connector 20. The step of connecting the upper member 16 and the lower member 14 together may include: securing a first rubber band to the first peg 18 of the upper member 16 and the first peg 18 of the lower member 14; and securing a second rubber band to the second peg 18 of the upper member 16 and the second peg 18 of the lower member 14. The method may further include placing a CPAP mask 22 over a portion of the face and falling asleep. The connected upper and lower members 14, 16 maintain facial integrity so that the CPAP mask 22 does not dislodge and thereby leak. The members 14, 16 may be stored on a storage base 12 when not in use. The storage base 12 may be a mold of the user's teeth.

A method of making 30 the present invention may include the following. Take the 3D Scan of the user's teeth. 3D Print the maxillary and mandibular model. Vacuum form Bitesplint material and add band buttons (pegs) to hold the appliance in place to keep the jaw shut. The appliance is custom fit using a 3D scan and 3D printed model to Vacu Form the appliance. Band buttons (pegs) are applied to the appliance to use rubber bands to keep the appliance secure and keep the mandible in place during sleep. When the appliance is in place the patient will get the facial structure needed to keep their CPAP mask fitting just as well as it did when they went to sleep keeping the facial integrity.

It should be understood, of course, that the foregoing relates to exemplary embodiments of the invention and that modifications may be made without departing from the spirit and scope of the invention as set forth in the following claims.

What is claimed is:

1. A facial integrity oral appliance comprising:
an upper member and a lower member, each of the upper member and lower member comprising:
a plurality of tooth impressions for receiving respective teeth of a dentition and having a facial surface on one side thereof and a lingual surface on the opposite side thereof, and
a connector connecting the upper member and the lower member together, wherein the connector comprises a rubber elasticity.

2. The appliance of claim 1, wherein each of the upper member and the lower member comprises a front portion for receiving incisal teeth and side portions for receiving occlusal teeth.

3. The appliance of claim 2, wherein each of the upper member and the lower member comprises:
a first peg comprising an end flange protruding from the facial surface of one of the side portions; and
a second peg comprising an end flange protruding from the facial surface of the other of the side portions.

4. The appliance of claim 3, wherein the connector comprises a first rubber band and a second rubber band, wherein the first rubber band connects to the first peg of the upper member and the first peg of the lower member and the second rubber band connects to the second peg of the upper member and the second peg of the lower member.

5. A method of maintaining facial integrity while sleeping comprising the steps of:
placing an upper member on upper teeth, wherein the upper member comprises a plurality of tooth impressions and having a facial surface on one side thereof and a lingual surface on the opposite side thereof;
placing a lower member on lower teeth, wherein the lower member comprises a plurality of tooth impressions and having a facial surface on one side thereof and a lingual surface on the opposite side thereof;
connecting the upper member and the lower member together by a connector comprising rubber elasticity.

6. The method of claim 5, wherein each of the upper member and the lower member comprises:
a first peg comprising an end flange protruding from the facial surface of a first side portion; and
a second peg comprising an end flange protruding from the facial surface of a second side portion opposite the first side portion.

7. The method of claim 6, wherein the step of connecting the upper member and the lower member together comprises:
securing a first rubber band to the first peg of the upper member and the first peg of the lower member; and
securing a second rubber band to the second peg of the upper member and the second peg of the lower member.

8. The method of claim 5, further comprising the step of:
placing a continuous positive airway pressure mask over a portion of a face.
